(19) United States
(12) Patent Application Publication
(10) Pub. No.: US 2024/0410797 A1
(43) Pub. Date: Dec. 12, 2024

(54) MICROSCOPIC OBJECT COLLECTING METHOD AND MICROSCOPIC OBJECT COLLECTING SYSTEM

(71) Applicants: University Public Corporation Osaka, Osaka-shi, Osaka (JP); Murata Manufacturing Co., Ltd., Nagaokakyo-shi, Kyoto-fu (JP)

(72) Inventors: Takuya IIDA, Sakai-shi (JP); Shiho TOKONAMI, Sakai-shi (JP); Mamoru TAMURA, Sakai-shi (JP); Kota HAYASHI, Sakai-shi (JP); Tsutomu YAMASAKI, Nagaokakyo-shi (JP); Hirohito WASHIDA, Nagaokakyo-shi (JP); Hiroki ISHIKAWA, Nagaokakyo-shi (JP)

(21) Appl. No.: 18/690,527

(22) PCT Filed: Sep. 8, 2022
(86) PCT No.: PCT/JP2022/033754

§ 371 Date: Mar. 8, 2024

(30) Foreign Application Priority Data
Sep. 10, 2021 (JP) 2021-147684

(51) Int. Cl. G01N 1/40

(52) U.S. Cl. CPC G01N 1/4022

(57) ABSTRACT

A microscopic object collecting method collects a plurality of microscopic objects dispersed in a sample. The method includes: irradiating a thin film with a plurality of laser beams, the thin film being provided on a bottom surface of an collection container containing the sample, the plurality of laser beams being separated from each other; and heating the sample with the plurality of laser beams to generate a plurality of microbubbles corresponding to the plurality of laser beams and to generate heat convection in the sample. An interval between adjacent two laser beams of the plurality of laser beams is narrower than a distance that allows three larger microbubbles to be virtually arranged in a gap between two microbubbles corresponding to the two laser beams, each of the three larger microbubbles being the larger one of the two microbubbles.

### TECHNICAL FIELD

The present disclosure relates to a microscopic object collecting method and a microscopic object collecting system, and more specifically, to a technology for collecting a plurality of microscopic objects dispersed in a liquid.

### BACKGROUND ART

A technique for collecting a plurality of microscopic objects (microparticles, cells, microorganisms, etc.) dispersed in a liquid has been proposed. For example, a device for collecting microscopic objects disclosed in WO 2018/159706 A (PTL 1) includes a light source and a container capable of holding a dispersion liquid in which a plurality of microscopic objects are dispersed. The container has a bottom surface on which a photothermal conversion member that converts light from the light source into heat is provided, and an inner surface on which immersion wetting occurs due to the dispersion liquid when the container comes into contact with the dispersion liquid. The photothermal conversion member generates heat convection in the dispersion liquid by heating the dispersion liquid. The inner surface generates Marangoni convection at a gas-liquid interface which is an interface between the dispersion liquid and a gas around the dispersion liquid.

### CITATION LIST

### Patent Literature

PTL 1: WO 2018/159706 A

### SUMMARY OF INVENTION

### Technical Problem

A collecting system that collects a plurality of microscopic objects dispersed in a liquid by light irradiation is desired to collect more microscopic objects in a shorter period of time, in other words, to collect microscopic objects with higher efficiency.

The present disclosure has been made to solve the above problems, and an object of the present disclosure is to provide a technology capable of highly efficiently collecting a plurality of microscopic objects dispersed in a liquid.

### Solution to Problem

(1) A microscopic object collecting method according to an aspect of the present disclosure collects a plurality of microscopic objects dispersed in a liquid. The method includes: irradiating a photothermal conversion region with a plurality of beams, the photothermal conversion region being provided on a bottom surface of a container containing the liquid, the plurality of beams being separated from each other; and heating the liquid with the plurality of beams to generate a plurality of microbubbles corresponding to the plurality of beams and to generate heat convection in the liquid. An interval between adjacent two beams of the plurality of beams is narrower than a distance that allows three larger microbubbles to be virtually arranged in a gap between two microbubbles corresponding to the two beams, each of the three larger microbubbles being the larger one of the two microbubbles.

(2) The interval is narrower than a distance that allows one of the three larger microbubbles to be virtually arranged in the gap.

(3) The distance between an irradiation position on the photothermal conversion region with each of the plurality of beams and a side wall of the container is longer than a diameter of a corresponding one of the plurality of microbubbles.

(4) The thermal conductivity of the side wall of the container is larger than a thermal conductivity of the liquid.

(5) An irradiation area on the photothermal conversion region with each of the two beams is larger than a contact area between a corresponding one of the two microbubbles and the photothermal conversion region.

(6) The method further includes: storing the liquid in the container, prior to the irradiating, such that a gas-liquid interface between the liquid and a surrounding gas is flat.

(7) The microscopic object collecting system according to another aspect of the present disclosure collects a plurality of microscopic objects dispersed in a liquid. The system includes: a holder that holds a storage container for the liquid, the storage container having a bottom surface provided with a photothermal conversion region; a light source that emits a plurality of beams; and an optical system that is configured to irradiate the photothermal conversion region with the plurality of beams that are separated from each other. By heating the liquid with the plurality of beams, a plurality of microbubbles corresponding to the plurality of beams are generated and heat convection is generated in the liquid. The optical system is configured such that an interval between adjacent two beams of the plurality of beams is narrower than a distance that allows three larger microbubbles to be virtually arranged in a gap between two microbubbles corresponding to the two beams, each of the three larger microbubbles being the larger one of the two microbubbles.

### Advantageous Effects of Invention

According to the present disclosure, a plurality of microscopic objects dispersed in a liquid can be collected with high efficiency.

### BRIEF DESCRIPTION OF DRAWINGS

FIG. 1 is a diagram schematically illustrating an example of an overall configuration of a microscopic object collecting system according to a first embodiment of the present disclosure.

FIG. 2 is a diagram schematically illustrating a configuration of a collection container and a laser module.

FIG. 3 is an image taken by photographing an appearance of the collection container.

FIG. 4 is a flowchart showing an example of a microscopic object collecting method according to this embodiment.

FIG. 5 is a diagram schematically illustrating another example of the overall configuration of the microscopic object collecting system according to the first embodiment.

FIG. 6 is a diagram schematically illustrating another example of a configuration of a laser device.

FIG. 7 is a flowchart showing another example of the microscopic object collecting method according to this embodiment.

FIG. 8 is a diagram for illustration of a microscopic object collecting mechanism.

FIG. 9 is a graph showing an example of a collection result of resin beads at the time of irradiation with a plurality of laser beams.

FIG. 10 is a diagram illustrating a simulation model used for fluid simulation.

FIG. 11 is a top view of the simulation model illustrated in FIG. 10.

FIG. 12 is a first view showing a fluid simulation result of heat convection at each pitch.

FIG. 13 is a second view showing a fluid simulation result of heat convection at each pitch.

FIG. 14 is a graph showing a simulation result of an average flow velocity of heat convection at each pitch.

FIG. 15 is a graph showing an actual measurement result of a collected number of resin beads at each pitch.

FIG. 16 is a diagram for illustration of a gap between two microbubbles generated at positions of adjacent heat sources at each pitch.

FIG. 17 is a view illustrating a fluid simulation result of heat convection around the microbubbles.

FIG. 18 is a graph showing a simulation result and an actual measurement result regarding a temperature of a side wall for each material of the side wall of the collection container.

FIG. 19 is a graph showing a simulation result regarding a flow velocity of heat convection for each material of the side wall of the collection container.

FIG. 20 is a graph showing an actual measurement result of the collected number of resin beads.

FIG. 21 is a diagram showing a simulation model for examining an influence of a shape and a height of the gas-liquid interface.

FIG. 22 is a view illustrating a fluid simulation result regarding the flow velocity of the heat convection.

FIG. 23 is a graph showing a fluid simulation result regarding a flow rate of the heat convection.

FIG. 24 is a graph for comparing flow rates of the heat convection between a flat liquid level and a curved liquid level.

FIG. 25 is an image taken by photographing the flat liquid level and the curved liquid level.

FIG. 26 is a graph showing an example of measurement results of the collected number of resin beads and collection efficiency on the flat liquid level.

FIG. 27 is a graph showing an example of measurement results of the collected number of resin beads and collection efficiency on the curved liquid level.

### DESCRIPTION OF EMBODIMENTS

Embodiments of the present disclosure will now be described in detail with reference to the drawings. The same or corresponding parts in the drawings are denoted by the same reference signs, and the description thereof will not be repeated.

### DEFINITION OF TERMS

In the present disclosure, the wording “order of nanometers” includes a range from 1 nm to 1000 nm (=1 μm). The wording “order of micrometers” includes a range from 1 μm to 1000 μm (=1 mm). Therefore, the wording “order ranging from nanometers to micrometers” indicates a range from 1 nm to 1000 μm, and typically indicates a range from several ten nm to several hundred μm, preferably indicates a range from 100 nm to 100 μm, and more preferably indicates a range from 1 μm to several tens of μm.

In the present disclosure, the term “microscopic object” means an object having a size on the order ranging from nanometers to micrometers. The shape of the microscopic object is not particularly limited, and examples of the shape include a spherical shape, an ellipsoidal shape, a rod shape, and the like. When the microscopic object has an ellipsoidal shape, at least one of the length in the minor axis direction and the length in the major axis direction of the ellipsoid may be on the order ranging from nanometers to micrometers. When the microscopic object has a rod shape, at least one of the width and the length of the rod may be on the order ranging from nanometers to micrometers.

Examples of the microscopic object include a metallic nanoparticle, a metallic nanoparticle aggregate, a metallic nanoparticle assembly structure, a semiconductor nanoparticle, an organic nanoparticle, a resin bead, a particulate matter (PM), and a nanodiamond. The “metallic nanoparticle” refers to a metallic particle having a size on the order of nanometers. The “metallic nanoparticle aggregate” refers to an aggregate formed by aggregation of a plurality of metallic nanoparticles. The “metallic nanoparticle assembly structure” refers to, for example, a structure in which a plurality of metallic nanoparticles is fixed to the surface of a base material (resin bead or the like) with an interaction site interposed therebetween, and are arranged at intervals less than or equal to the diameter of the metallic nanoparticles with gaps therebetween. The “semiconductor nanoparticle” refers to a semiconductor particle having a size on the order of nanometers. The “organic nanoparticle” refers to a particle made of an organic compound having a size on the order of nanometers. The “resin bead” refers to a particle made of a resin having a size on the order ranging from nanometers to micrometers. The “PM” refers to a particulate substance having a size on the order of micrometers. Examples of the PM include PM 2.5 and suspended particulate matter (SPM).

The microscopic object may be a substance derived from a living body (biological substance). More specifically, the microscopic object may include cells, microorganisms (such as bacteria or fungi), biopolymers (such as proteins, nucleic acids, lipids, or polysaccharides), antigens (such as allergens), and viruses.

In the present disclosure, “microbubbles” mean bubbles having a size (diameter) on the order of micrometers.

In the following description, an x direction and a y direction represent the horizontal direction. The x direction and the y direction are orthogonal to each other. A z direction represents a vertical direction. The direction of gravity is downward in the z direction. The upward in the z direction is abbreviated as “upper”, and the downward in the z direction is abbreviated as “lower”.

### First Embodiment

### <System Configuration>

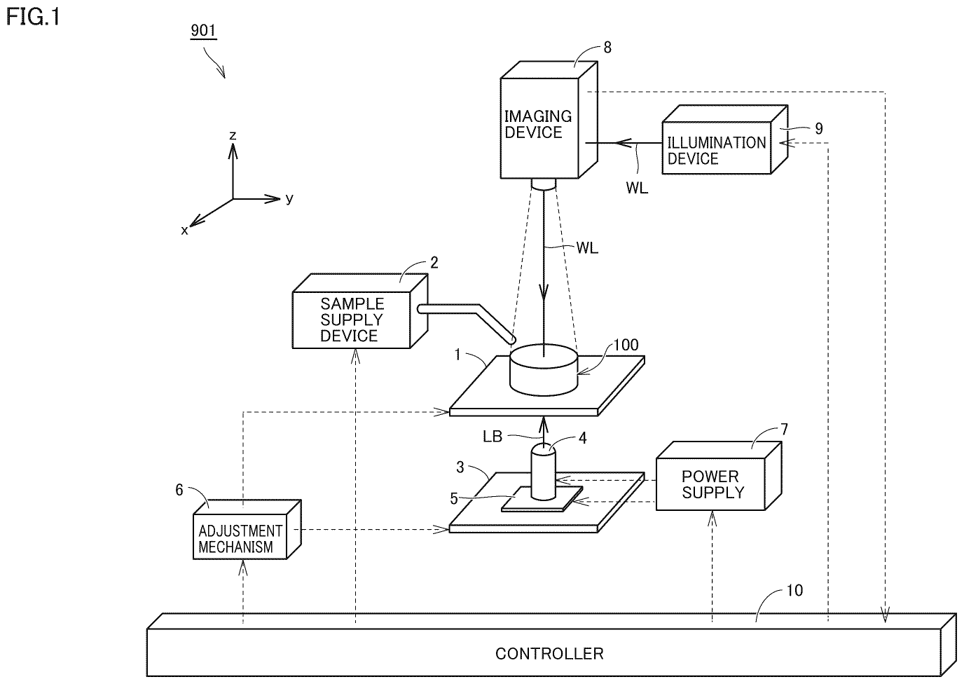

FIG. 1 is a diagram schematically illustrating an example of an overall configuration of a microscopic object collecting system according to a first embodiment of the present disclosure. A collecting system 901 includes a sample stage 1, a sample supply device 2, a light source stage 3, a laser module 4, a cooling device 5, an adjustment mechanism 6, a power supply 7, an imaging device 8, an illumination device 9, and a controller 10.

Sample stage 1 holds a collection container 100 containing a liquid sample. Sample stage 1 is, for example, an xyz-axis stage, and is configured to move in the x direction, the y direction, and the z direction. Sample stage 1 corresponds to a “holder” according to the present disclosure.

Sample supply device 2 supplies a sample to collection container 100 in response to a command from controller 10. As sample supply device 2, for example, a dispenser can be used.

Light source stage 3 holds laser module 4 and cooling device 5. Light source stage 3 is, for example, an XYZ-axis stage, and is configured to move in the x direction, the y direction, and the z direction. In this example, light source stage 3 is disposed below sample stage 1.

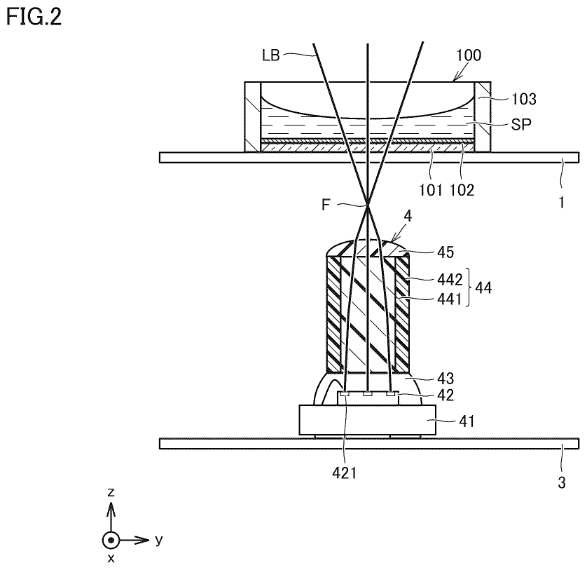

Laser module 4 is a laser light source such as a semiconductor laser module, and emits a plurality of laser beams (indicated by L in the drawing) in response to a command from controller 10. A wavelength of the laser beams in this example is within the near infrared region, and is 850 nm, for example. A configuration of laser module 4 will be described in further detail with reference to FIG. 2.

Cooling device 5 cools laser module 4. Cooling device 5 is, for example, a Peltier element or a heat sink. By cooling laser module 4, it is possible to prevent a failure of laser module 4 due to a temperature rise of laser module 4, and to suppress a decrease in laser output. In FIG. 2 and subsequent drawings, cooling device 5 is not illustrated.

Adjustment mechanism 6 is configured to adjust positions of sample stage 1 in the x direction, the y direction, and the z direction and to adjust positions of light source stage 3 in the x direction, the y direction, and the z direction in response to a command from controller 10. As a result, adjustment mechanism 6 is able to adjust a relative positional relationship between collection container 100 mounted on sample stage 1 and laser module 4 installed on light source stage 3. However, for example, adjustment mechanism 6 may adjust a position of collection container 100 with respect to laser module 4 that is fixed, or may adjust a position of laser module 4 with respect to collection container 100 that is fixed. In the example described below, when a light irradiation position is set, the position of sample stage 1 in the horizontal direction (the positions in the x direction and the y direction) is adjusted, and a height of light source stage 3 (the position in the z direction) is adjusted. Note that light source stage 3 and adjustment mechanism 6 correspond to an “optical system” according to the present disclosure.

Power supply 7 supplies a current for driving laser module 4 in response to a command from controller 10. In addition, power supply 7 supplies a current for driving cooling device 5 (such as a Peltier element or a fan of a heat sink).

In response to a command from controller 10, imaging device 8 takes an image of the sample in collection container 100 and outputs the taken image to controller 10. As imaging device 8, a video camera including a charge coupled device (CCD) image sensor, a complementary metal-oxide-semiconductor (CMOS) image sensor, or the like may be used.

Illumination device 9 emits illumination light (for example, white light indicated by WL) for illuminating the sample on collection container 100 in response to a command from controller 10. For example, a white light emitting diode (LED), a halogen lamp, or the like can be used as illumination device 9. Note that imaging device 8 and illumination device 9 are devices merely for observing a state of the sample, and are not essential components for collecting microscopic objects by collecting system 901.

Controller 10 is a microcomputer, for example, including a processor such as a central processing unit (CPU), a memory such as a read only memory (ROM) and a random access memory (RAM), and an input/output port (these components are not illustrated). Controller 10 controls each device (sample supply device 2, adjustment mechanism 6, power supply 7, imaging device 8, and illumination device 9) constituting collecting system 901.

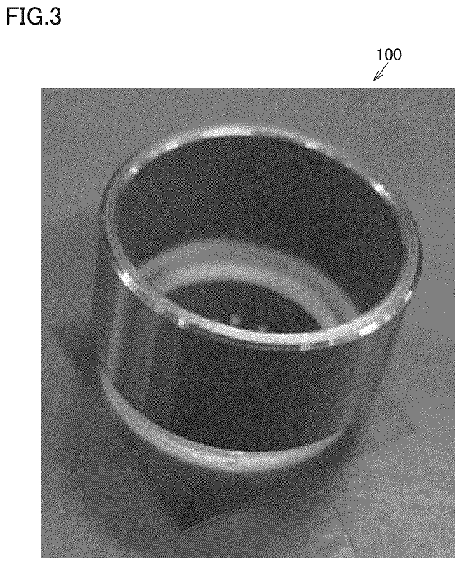

FIG. 2 is a diagram schematically illustrating a configuration of collection container 100 and laser module 4. FIG. 3 is an image taken by photographing an appearance of collection container 100. Referring to FIGS. 2 and 3, collection container 100 has a cylindrical shape with an open top, for example, and includes a substrate 101, a thin film 102, and a side wall 103.

Substrate 101 is disposed on a bottom surface of collection container 100. Substrate 101 is made of a material that is transparent to the laser beams (near-infrared light) from laser module 4 and transparent to the illumination light (white light) from illumination device 9. Examples of such a material include glass, quartz, and silicone.

Thin film 102 is disposed on substrate 101. Thin film 102 absorbs the laser beams from laser module 4 and converts light energy into thermal energy. A material of thin film 102 preferably has high photothermal conversion efficiency in the wavelength region (near-infrared region in this example) of a laser beam. In this example, a gold thin film is provided as thin film 102. The gold thin film can be formed using a known method such as sputtering or electroless plating. A thickness (film thickness) of thin film 102 is determined as a design or experimentally in consideration of the laser output and an absorption wavelength range and a photothermal conversion efficiency of the material of thin film 102, and is typically on the order of nanometers (for example, 10 nm).

Free electrons on a surface of thin film 102 form surface plasmons and are oscillated by the laser beam. This causes polarization. The energy of this polarization is converted into energy of lattice vibration by the Coulomb interaction between the free electron and the atomic nucleus. As a result, thin film 102 generates heat. In the following, this effect is referred to as a “photothermal effect”.

The material of thin film 102 is not limited to gold, and may be a metal element other than gold (for example, silver) that can produce a photothermal effect, a metallic nanoparticle assembly structure (for example, a structure using gold nanoparticles or silver nanoparticles), or the like. The material of thin film 102 may be a material other than metal having a high light absorption rate in the wavelength range of the laser beam. Examples of such a material include a material close to a black body (for example, a carbon nanotube black body).

Note that thin film 102 may not be formed on the entire surface of substrate 101, and may be formed on at least a part of substrate 101. A region where thin film 102 is formed corresponds to a “photothermal conversion region” according to the present disclosure.

Side wall 103 has a hollow cylindrical shape in this example. Optically, a material of side wall 103 is not particularly limited, but preferably has good heat dissipation characteristics (high thermal conductivity) as described later. A material of side wall 103 in the present embodiment is a steel use stainless (SUS).

Laser module 4 includes a base material 41, a surface emitting element 42, a bonding member 43, an optical waveguide 44, and a lens 45.

Base material 41 is a flat plate made of an insulating material, and is, for example, a printed wiring board or a ceramic substrate. Surface emitting element 42 is mounted on the surface of base material 41. Base material 141 supplies a drive current from power supply 7 (see FIG. 1) to surface emitting element 42.

Surface emitting element 42 is an array-type vertical cavity surface emitting laser (VCSEL), for example. Surface emitting element 42 has a plurality of light emitting regions 421 (nine in the example described later). The plurality of light emitting regions 421 is arranged in a two-dimensional array. All light emitting regions 421 simultaneously emit light, and each of the plurality of light emitting regions 421 emits a laser beam (indicated by LB). Each laser beam is emitted in a direction perpendicular to the surface of surface emitting element 42 (the upward in the z direction).

Bonding member 43 is, for example, an adhesive, and bonds optical waveguide 44 onto surface emitting element 42. Bonding member 43 is made of a material transparent to light (near-infrared light) emitted from surface emitting element 42.

Optical waveguide 44 converges the plurality of laser beams emitted from surface emitting element 42. The material of optical waveguide 44 is transparent to light emitted from surface emitting element 42, and is, for example, resin or glass. Optical waveguide 44 includes a core 441 and a cladding 442. Core 441 has a cylindrical shape. An incident end of core 441 is configured to cover light emitting regions 421 as a whole so that all the laser beams emitted from surface emitting element 42 are incident. Cladding 442 has a hollow cylindrical shape. Cladding 442 is provided so as to cover a side surface of core 441.

Lens 45 is a plano-convex lens and has a flat surface and a convex surface. A plane of lens 45 is bonded to the output end of optical waveguide 44. The convex surface of lens 45 protrudes from a laser emission portion of laser module 4 in the light emission direction.

A propagation path of the laser beam in laser module 4 will be described. Optical waveguide 44 is a graded index (GI) type optical fiber. Therefore, the refractive index of core 441 of optical waveguide 44 is the highest at the radial center of core 441, and smoothly decreases toward the outside in the radial direction. The laser beam propagating inside core 441 has a plurality of modes having different propagation distances. Light of a low-order mode travels through the center of the core, and light of a high-order mode travels through a region outside the center of the core. The propagation distance of the light of the low-order mode is short, but the propagation speed of the light of the low-order mode is relatively low due to the high refractive index at the center of the core. On the contrary, in the light of the high-order mode, the propagation distance is long, but the propagation speed is relatively high. The refractive index distribution of core 441 is designed such that a difference in propagation time between the modes is sufficiently small.

The plurality of laser beams propagating inside core 441 having such a refractive index distribution forms a node and an antinode. Note that the positions of node and antinode may change according to the wavelength of a laser beam. Regarding the traveling direction of the laser beam, the length of optical waveguide 44 is determined so that the output end of optical waveguide 44 is not positioned in the middle from the node to the antinode. In other words, the length of optical waveguide 44 is determined such that the output end of optical waveguide 44 is located on the way from the antinode to the node, or the output end of optical waveguide 44 coincides with the antinode. As a result, the plurality of laser beams propagated through optical waveguide 44 is emitted from the output end of optical waveguide 44 in such a manner that they are converged. The plurality of emitted laser beams is further converged by lens 45 to constitute an identical focal point F. Collection container 100 on sample stage 1 is irradiated with a plurality of laser beams emitted upward from laser module 4.

The plurality of laser beams emitted upward from the tip of laser module 4 is separate near lens 45, but intersects each other above lens 45 to form focal point F. Then, the plurality of laser beams is separated again above focal point F. As described with reference to FIG. 1, controller 10 is able to adjust the height of light source stage 3 by controlling adjustment mechanism 6. Therefore, when the height of light source stage 3 is adjusted such that focal point F is located on the bottom surface of collection container 100, single irradiation to collection container 100 is realized (single irradiation method). On the other hand, when the height of light source stage 3 is adjusted such that focal point F is located below the bottom surface of collection container 100, multiple irradiation to collection container 100 is realized (multiple irradiation method). In this manner, controller 10 is able to switch between the single irradiation method and the multiple irradiation method.

When the multiple irradiation method is selected, a plurality of laser spots is located on the bottom surface (thin film 102) of collection container 100. An interval between the centers of these laser spots is referred to as “pitch”. The pitch increases as the position of the bottom surface of collection container 100 moves away from focal point F. Therefore, controller 10 can set the pitch to a desired value by controlling adjustment mechanism 6.

### <Collecting Flow>

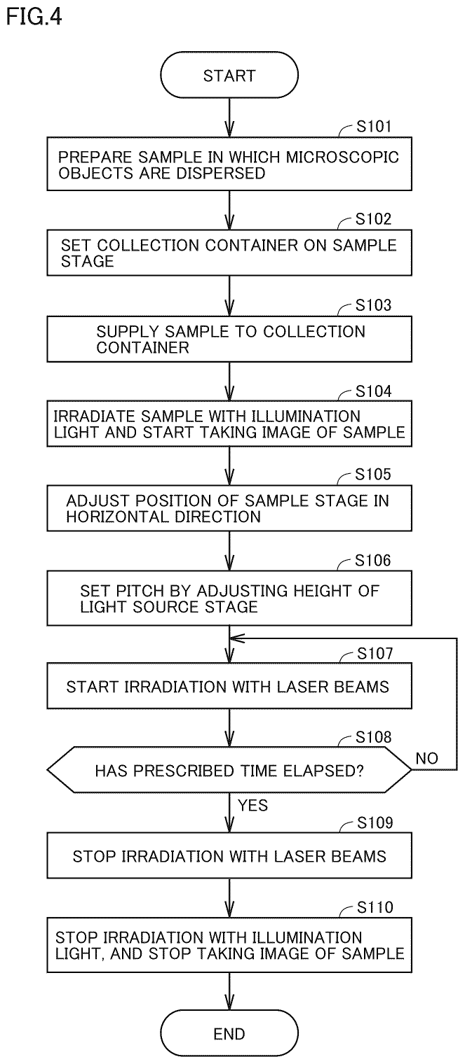

FIG. 4 is a flowchart showing an example of a microscopic object collecting method according to this embodiment. In this flowchart, each step after step S103 is basically achieved by software processing by controller 10, but a part or all of the steps may be achieved by hardware (electric circuit) in controller 10. Hereinafter, the step is abbreviated as S.

In S101, a sample in which microscopic objects are dispersed in a dispersion medium is prepared by a measurer. The prepared sample is stored in sample supply device 2.

In S102, controller 10 sets collection container 100 on sample stage 1. This process can be realized by, for example, a feed mechanism (not illustrated) provided in collecting system 901.

In S103, controller 10 supplies an appropriate amount of sample to collection container 100 by controlling sample supply device 2. A supply amount of the sample may be, for example, a trace amount of about several tens μL to several hundreds μL, or may be a larger amount (1 mL in the example described later). By using collection container 100 provided with side wall 103 as in the present embodiment, a larger amount of sample can be accommodated in collection container 100 as compared with the case of using a flat substrate (not illustrated).

In S104, controller 10 controls illumination device 9 to start irradiation of the sample with illumination light (white light). In addition, controller 10 controls imaging device 8 to start taking an image of the sample. Note that a process of S104 is a process for observing the sample, and is not a process for collecting microscopic objects. In the process of S104, fluorescence observation of the sample may be performed.

In S105, controller 10 adjusts the position of sample stage 1 in the horizontal direction by controlling adjustment mechanism 6. As a result, a target position in the sample can be irradiated with a plurality of laser beams from laser module 4. More specifically, controller 10 can acquire a position of the sample in the horizontal direction by extracting an outer shape pattern of the sample from the image captured by imaging device 8 using the pattern recognition image processing technique. Then, controller 10 appropriately adjusts the position of light source stage 3 in the horizontal direction from the initial position, so that the irradiation positions of the plurality of laser beams in the horizontal direction can be aligned with target positions in the sample.

In S106, controller 10 controls adjustment mechanism 6 to adjust the height of light source stage 3. As a result, it is possible to switch between the single irradiation method and the multiple irradiation method, and to adjust the pitch in the multiple irradiation method. A position in the vertical direction of focal point F where all laser beams are collected is known from the specification (wavelength of the laser beam, shapes of optical waveguide 44 and lens 45, and the like) of laser module 4. Therefore, controller 10 is able to switch between the single irradiation method and the multiple irradiation method by appropriately adjusting the height of light source stage 3 from the initial height. In addition, controller 10 is able to set the pitch to a desired value by storing a correspondence relationship between the height of light source stage 3 and the pitch in the memory in advance.

In S107, controller 10 controls power supply 7 so that the irradiation of the sample with the plurality of laser beams from laser module 4 is started. Controller 10 then determines whether a prescribed time has elapsed since the laser beam was emitted (S108). The prescribed time is, for example, about several 10 seconds to several minutes, and is set by the measurer according to the type of the microscopic objects, the type of the dispersion medium, the volume of the dispersion medium, the concentration of the microscopic objects in the dispersion medium, and the like. Controller 10 continues the irradiation with the laser beam until the prescribed time elapses (NO in S108). Along with this light irradiation, the microscopic objects are collected according to the mechanism described later. When the prescribed time elapses (YES in S108), controller 10 controls power supply 7 such that the irradiation of the sample with the plurality of laser beams from laser module 4 is stopped (S109). In addition, controller 10 controls illumination device 9 to stop the irradiation of the sample with the illumination light, and controls imaging device 8 to stop taking an image of the sample (S110). Thus, a series of processes ends.

### <Another Example of Laser Configuration>

In FIGS. 1 to 4, the configuration in which a large number of laser beams are output from one laser module 4 has been described as an example, but a laser device including a plurality of laser modules may be provided.

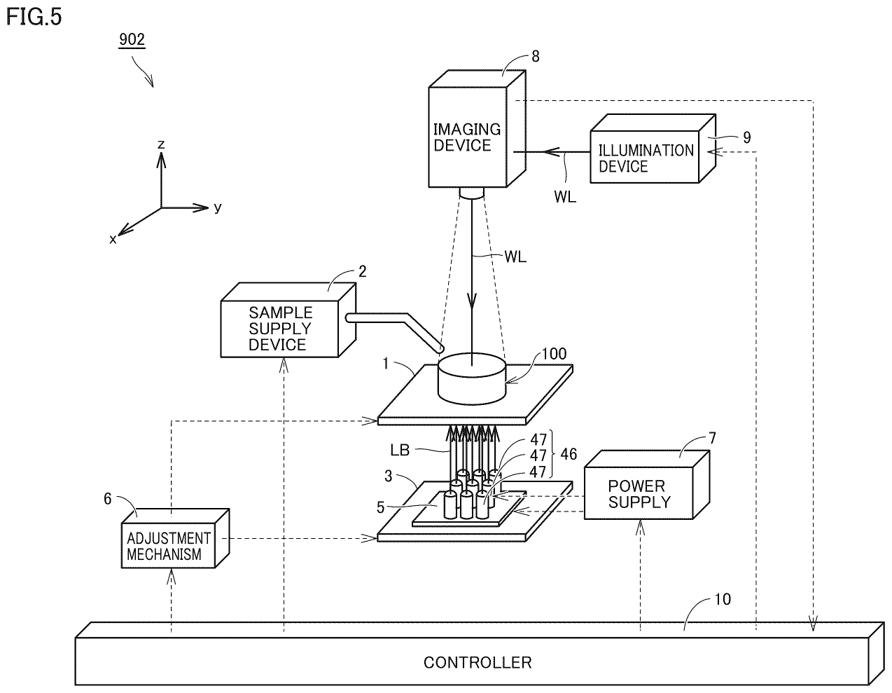

FIG. 5 is a diagram schematically illustrating another example of the overall configuration of the microscopic object collecting system according to the first embodiment. A collecting system 902 is different from collecting system 901 (see FIG. 1) in that a laser device 46 is provided instead of laser module 4. Laser device 46 includes a plurality of laser modules 47. FIG. 4 illustrates an example in which the number of laser modules 47 is 9, but the number of laser modules 47 is not particularly limited as long as it is greater than or equal to 2. Each of laser modules 47 includes a plurality of surface emitting elements 42.

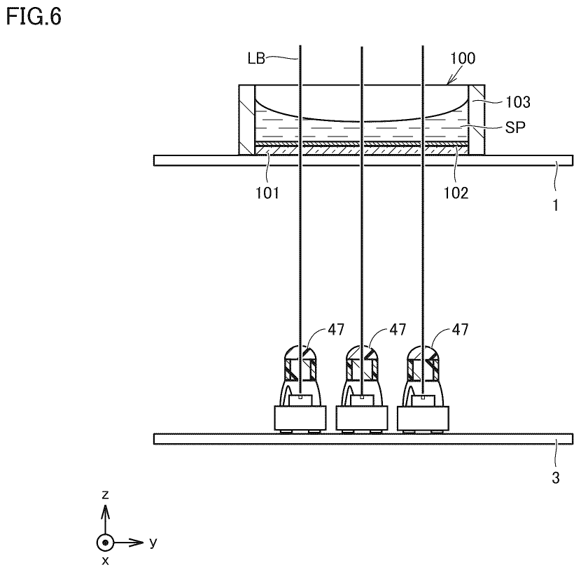

FIG. 6 is a diagram schematically illustrating another example of a configuration of the laser device. In this example, one laser beam is output from each laser module 47. In this case, various “pitches” (intervals between the laser spots) on the bottom surface of collection container 100 can be realized by changing intervals between adjacent laser modules 47.

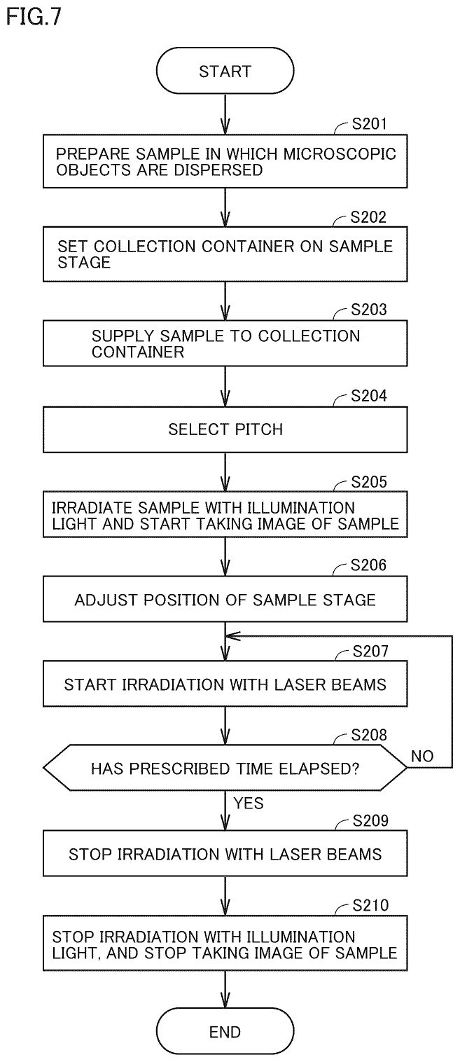

FIG. 7 is a flowchart showing another example of the microscopic object collecting method according to this embodiment. Since processes of S201 to S203 are similar to the processes of S101 to S103 of FIG. 4, the description will not be repeated.

In S204, controller 10 selects a pitch according to the laser output, the type of dispersion medium, and the like on the basis of a simulation result and/or an experimental result described later. For example, collecting system 902 may be configured such that laser device 46 is replaceable, and various laser devices 46 for replacement may be prepared in advance. Intervals between adjacent laser modules 47 are different between laser devices 46 for replacement. That is, these laser devices 46 correspond to different pitches. Laser device 46 may be attached to collecting system 902 to achieve a pitch selected by controller 10 (which may of course be selected by the measurer).

In S205, controller 10 controls illumination device 9 to start irradiation of the sample with illumination light (white light). In addition, controller 10 controls imaging device 8 to start taking an image of the sample. Thereafter, controller 10 adjusts the position of sample stage 1 in the horizontal direction by controlling adjustment mechanism 6 (S206). Controller 10 may adjust the height of light source stage 3 by controlling adjustment mechanism 6. Since processes of S207 to S210 are similar to the processes of S107 to S110 of FIG. 4, the description will not be repeated.

### <Collecting Mechanism>

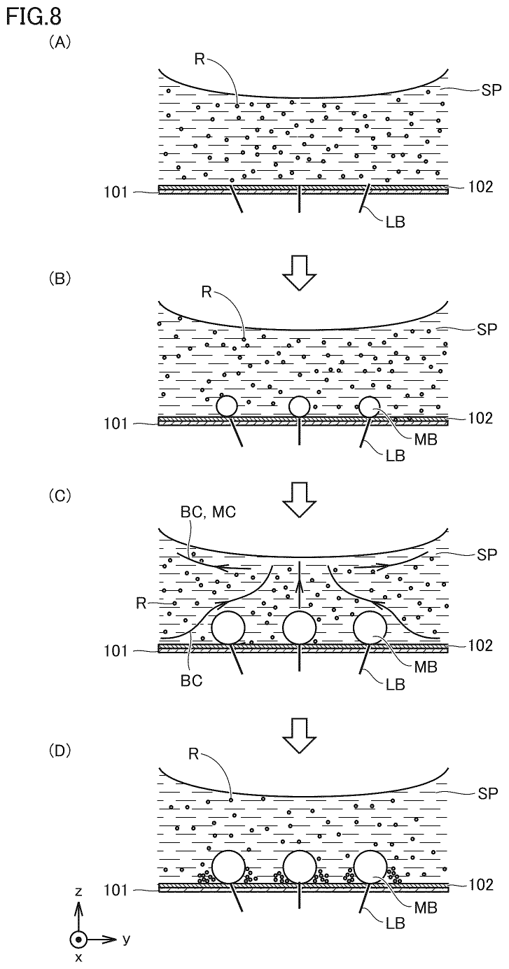

FIG. 8 is a diagram for illustration of a microscopic object collecting mechanism. This figure is provided in order to illustrate in detail a phenomenon that occurs during the processes of S107 and S108 of FIG. 4 or during the processes of S207 and S208 of FIG. 7. Here, in order to avoid complication of the drawing, a configuration in which the sample is irradiated with three laser beams will be described as an example.

Referring to FIG. 8(A), when light irradiation is started, a plurality of laser spots is locally heated by the photothermal effect of thin film 102. Then, as shown in FIG. 8(B), the dispersion medium (for example, ultrapure water) boils in the vicinity of each of the plurality of laser spots, and microbubbles (indicated by MB) are generated. Each of the microbubbles grows over time.

Along with the irradiation of the laser light, regular heat convection is constantly generated in addition to microbubbles in the dispersion medium. In FIG. 8(C), directions of heat convection are indicated by arrows. Heat convection is classified into buoyancy convection (indicated by BC) and Marangoni convection (indicated by MC).

The closer to the laser spot, the higher the temperature of the dispersion medium. That is, a temperature gradient is generated in the dispersion medium by light irradiation. This temperature gradient causes buoyancy convection. More specifically, the dispersion medium present above the region where the microbubbles are generated becomes relatively dilute by heating and rises by buoyancy. At the same time, a relatively low-temperature dispersion medium present in the horizontal direction of the microbubbles flows into the microbubbles.

In general, an interfacial tension generated on a bubble surface depends on a molecular density at the bubble surface. The higher the molecular density, the smaller the interfacial tension. The molecular density in the present embodiment is affected by not only densities of molecules constituting the dispersion medium but also densities of microscopic objects. Therefore, when there is a density gradient of the microscopic objects at the gas-liquid interface between the microbubble and the dispersion medium, a region where the density of the microscopic objects is high (usually lower region) is pulled toward a region where the density of the microscopic objects is low (upper region) so that the interfacial tension is balanced. The movement of the gas-liquid interface at this time is transmitted within the liquid (bulk), and Marangoni convection occurs. The Marangoni convection depends on the density gradient but does not depend on gravity.

The microscopic objects are transported towards the microbubbles by heat convection (buoyancy convection and/or Marangoni convection) and captured by the microbubbles. More specifically, a “stagnation region” that is a region where the flow velocity of the heat convection becomes substantially zero is generated around the microbubbles. As a result of the microscopic objects transported by the heat convection being captured in the stagnation region, the microscopic objects are collected in the vicinity of the laser spots (see FIG. 8(D)). In this manner, the microbubbles function as “stoppers” that block the microscopic objects, thereby becoming collection sites of the microscopic objects. An action of concentrating and collecting the microscopic objects dispersed in the sample in the vicinity of the laser spots according to the above mechanism can also be referred to as “optical condensation”.

Irradiation with a plurality of laser beams (multiple irradiation) causes fast convection toward a void between adjacent microbubbles. Due to the influence of this convection, a large number of microscopic objects are collected in the stagnation region generated between adjacent microbubbles. Therefore, when conditions such as laser output are the same for both the single irradiation and the multiple irradiation, the multiple irradiation tends to increase the accumulation amount of the microscopic objects.

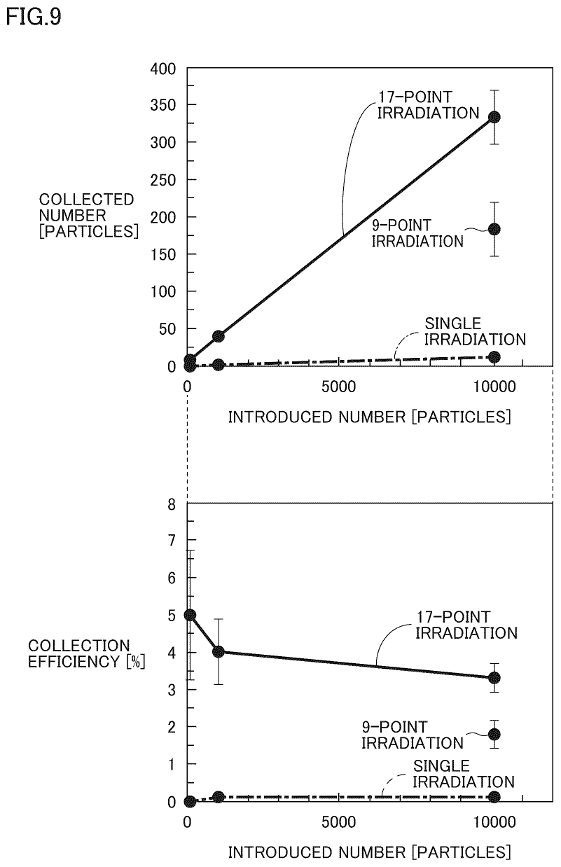

FIG. 9 is a graph showing an example of a collection result of resin beads at the time of irradiation with a plurality of laser beams. In this example, 9-point irradiation and 17-point irradiation were performed as the multiple irradiation, and the single irradiation was also performed as comparison. The horizontal axis represents the number of resin beads introduced into the sample (introduced number). The upper vertical axis represents the total number of resin beads (collected number) collected in all laser spots. The lower vertical axis represents the collection efficiency. The collection efficiency is the collected number with respect to the introduced number. For example, when the collected number is 500 with respect to the introduced number 10,000, the collection efficiency is calculated as 500/10000×100=5%.

It was confirmed that by performing multiple irradiation, the number of collected resin beads (collected number) was increased and the collection efficiency of the resin beads was also increased as compared with the case in which the single irradiation is performed. Specifically, when the introduced number was 10,000, the collected number 183 in the 9-point irradiation was 15.2 times of the collected number 12 in the single irradiation. From this, it can be seen that the collected number per microbubble in the 9-point irradiation was 1.69 times of the collected number in the single irradiation. In addition, when the introduced number was similarly 10,000, the collected number 333 in 17-point irradiation was 27.8 times of the collected number 12 in the single irradiation. From this, it can be seen that the collected number per microbubble in 17-point irradiation was 1.63 times of the collected number in the single irradiation.

### <Influence of Pitch>

The present inventors have studied the influence of the interval (pitch) between the laser spots at the position of the bottom surface of collection container 100 given to the collection efficiency of the microscopic objects. First, fluid simulation of heat convection in a case where the pitch is set to various values will be described.

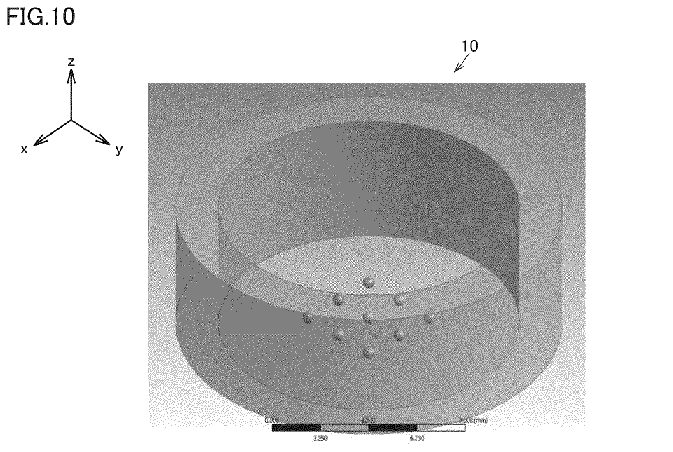

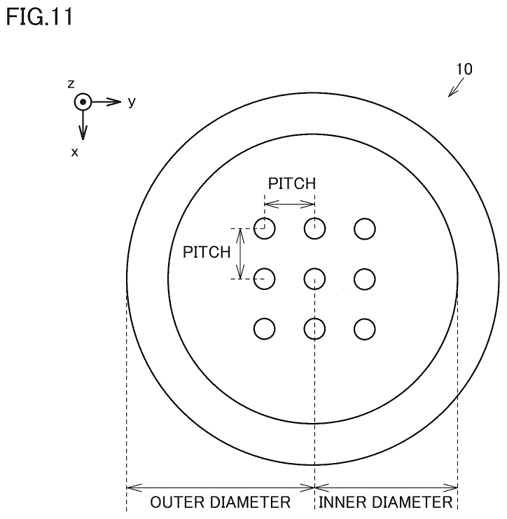

FIG. 10 is a diagram illustrating a simulation model used for the fluid simulation. FIG. 11 is a top view of the simulation model illustrated in FIG. 10. In this example, 9 heat sources were arranged on the bottom surface of collection container 100 in a 3×3 lattice pattern. Each of the 9 heat sources corresponds to a laser spot. The position of the central heat source among the 9 heat sources was matched with the center position of the circular bottom surface of collection container 100. An inner diameter and an outer diameter of cylindrical side wall 103 were 14 mm and 18 mm, respectively. A volume of the sample stored in collection container 100 was 1 mL.

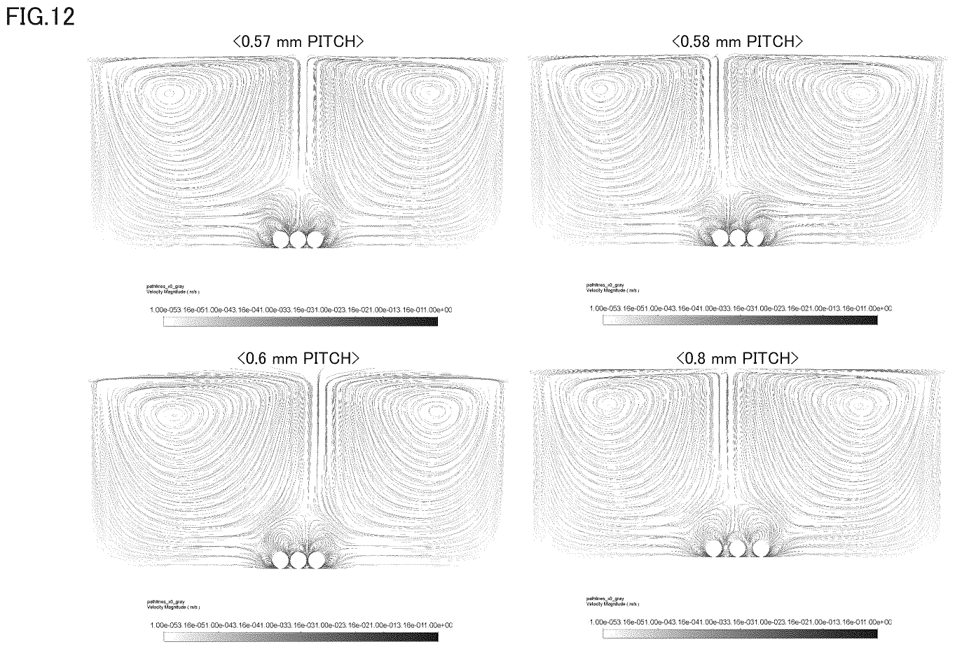

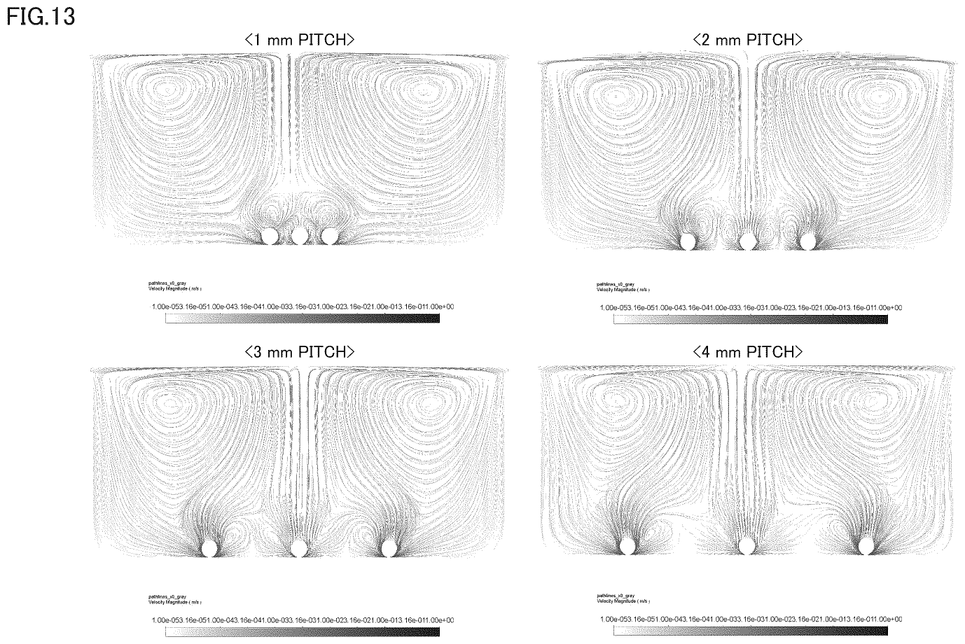

FIGS. 12 and 13 are views showing fluid simulation results of heat convection at each pitch. In this simulation, the pitch (interval between the centers of the heat sources) was set in eight ways of 0.57 mm, 0.58 mm, 0.6 mm, 0.8 mm, 1 mm, 2 mm, 3 mm, and 4 mm. A size of the microbubbles was set to 500 μm (=0.5 mm) in diameter. FIGS. 12 and 13 illustrate only a vertical cross section including three heat sources arranged in a line.

At any of the pitches, the heat convection rose from the three microbubbles (especially the microbubble at the center) towards the gas-liquid interface. The heat convection then flowed horizontally outward along the gas-liquid interface. The heat convection then fell down along side wall 103 back to three microbubbles. In collection container 100, the sample is circulated by the heat convection flowing in such a direction.

It can be understood that in the case where the pitch was extremely narrow, specifically, in the case where the pitch was less than or equal to 1 mm, the heat convection directed upward from the microbubble at the center among the three microbubbles and the heat convection directed downward from the microbubbles at both ends were inseparable. It can be understood that in addition, in the case where the pitch was 2 mm, although inseparability of this case was weaker than that in the case in which the pitch was less than or equal to 1 mm, the heat convection directed upward from the microbubble at the center and the heat convection directed downward from the microbubbles at both ends joined in a narrow range. As described above, in the case where the pitch was less than or equal to 2 mm, a synergistic effect in which the heat convection upward from the three microbubbles is integrated or strengthened was generated, and the flow velocity of the heat convection was faster than that in the case of the single irradiation.

On the other hand, in the case where the pitch was wide, specifically, in the case where the pitch was greater than or equal to 3 mm, the synergistic effect between the heat convection was weaker than that in the case where the pitch was less than or equal to 2 mm. In addition, the presence of a region where heat convection stagnated was remarkable in gaps between adjacent microbubbles. In this region, the heat convection swirled, and the flow velocity of the heat convection was low.

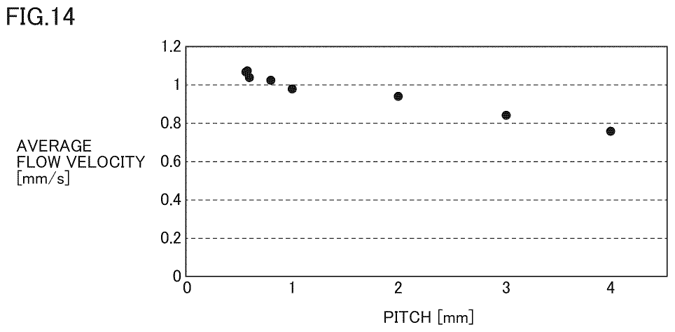

FIG. 14 is a graph showing a simulation result of an average flow velocity of the heat convection at each pitch. The horizontal axis represents the pitch, and the vertical axis represents the average flow velocity of the heat convection. The average flow velocity is obtained by calculating a local flow velocity for each region over the entire sample and obtaining an average value thereof. It was confirmed that the average flow velocity tended to increase as the pitch became narrower.

Next, a result when the resin beads as an example of the “microscopic objects” according to the present disclosure were actually collected will be described. In this example, polystyrene resin beads were used.

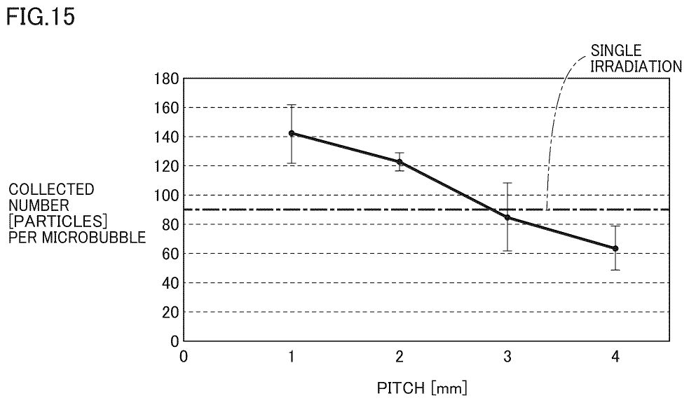

FIG. 15 is a diagram showing an actual measurement result of the collected number of resin beads at each pitch. The horizontal axis represents the pitch. The vertical axis represents the collected number of resin beads per microbubble. For comparison, the collected number of the resin beads collected in the microbubbles at the time of single irradiation is indicated by a one-dot chain line.

When the pitch was greater than or equal to 3 mm, the collected number of resin beads per macrobubble was smaller than the collected number of resin beads at the time of single irradiation. On the other hand, when the pitch was less than or equal to 2 mm, the collected number of resin beads per macrobubble was larger than the collected number of resin beads at the time of single irradiation. In particular, when the pitch was 1 mm, the collected number of resin beads per macrobubble was the largest. These actual measurement results are in good agreement with the fluid simulation results described with reference to FIGS. 12 and 13. Therefore, even in a case where the pitch is less than 1 mm, it is assumed that the collected number greater than or equal to that in the case where the pitch is 1 mm can be achieved.

As described above, in the present embodiment, when the size of the microbubbles is 0.5 mm in diameter, it is desirable to set the pitch to less than or equal to 2 mm, and it is more desirable to set the pitch to less than or equal to 1 mm. Here, at least within a range where the sample volume is small, the “scale law” in which a similar phenomenon occurs even when the scale is enlarged/reduced is established. Therefore, in the following, by using the size of microbubbles as a reference, the condition that it is desirable to set the pitch to less than or equal to 2 mm or less than or equal to 1 mm is generalized.

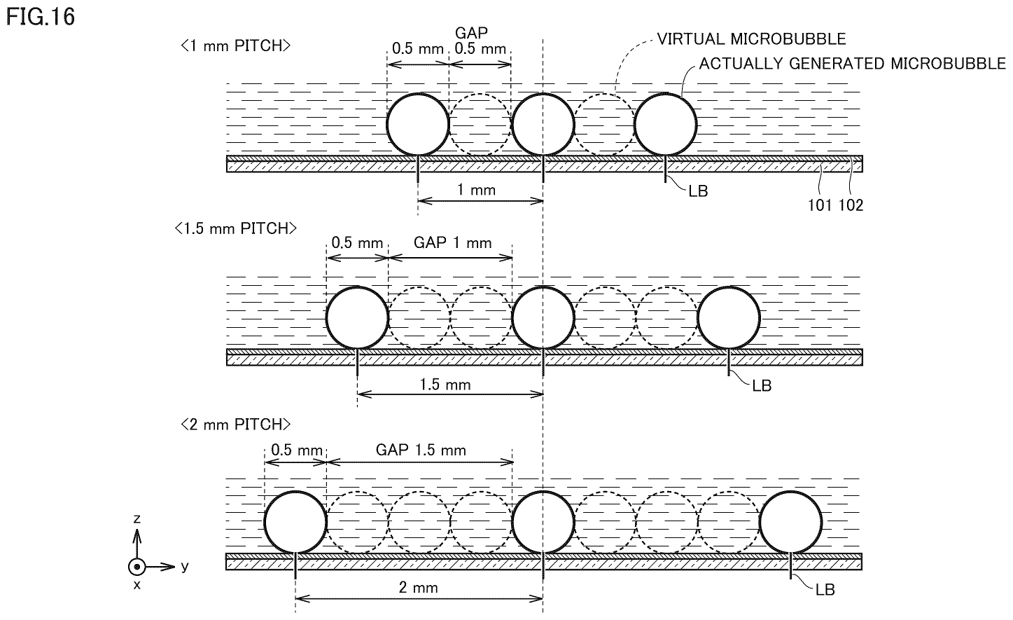

FIG. 16 is a diagram for illustration of a gap (space) between two microbubbles generated at positions of adjacent heat sources at each pitch.

When the pitch is 1 mm, a gap of 0.5 mm is generated between two adjacent microbubbles (microbubbles actually generated). The size of the gap is equal to the size (diameter) of the microbubbles. That is, one microbubble can be virtually arranged in the gap. Similarly, when the pitch is 1.5 mm, a gap of 1 mm is generated between two adjacent microbubbles. Two microbubbles can be virtually arranged in this gap. When the pitch is 2 mm, a gap of 1.5 mm is generated between two adjacent microbubbles. Three microbubbles can be virtually arranged in this gap.

In this example, for the sake of simplicity, the situation in which the sizes of all the microbubbles are equal has been described as an example, but the sizes of the microbubbles actually generated may vary to a certain extent. As the size of each microbubble virtually arranged, the size (diameter) of the larger one of the two microbubbles actually generated can be adopted. This is because, if the size of the smaller microbubbles is adopted, it cannot be denied that the heat convection is hindered due to excessive narrowing of gaps between adjacent microbubbles.

The condition that the pitch is set to less than or equal to 2 mm can be rephrased as a condition that “the pitch is narrower than a distance that allows three larger microbubbles to be virtually arranged in a gap between two microbubbles corresponding to the two beams, each of the three larger microbubbles being the larger one of the two microbubbles”. The condition that the pitch is set to less than or equal to 1 mm can be rephrased as a condition that “the pitch is narrower than a distance that allows one of the three larger microbubbles to be virtually arranged in the gap”.

### <Distance Between Microbubble and Side Wall>

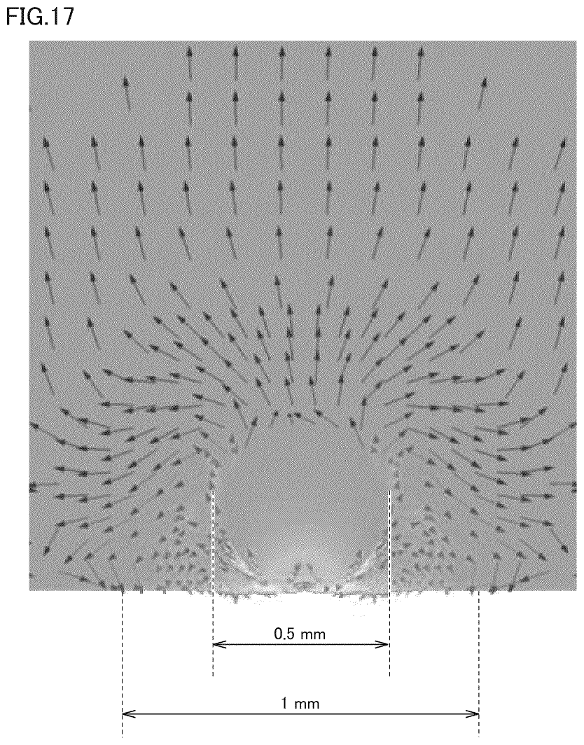

FIG. 17 is a view illustrating a fluid simulation result of heat convection around the microbubbles. FIG. 17 shows that particularly strong heat convection occurs within a range of 1 mm or less from the heat source (laser spot). Therefore, from the viewpoint of effectively utilizing heat convection, it is desirable that the position of side wall 103 of collection container 100 is outside this range. That is, it is desirable to design the size and/or shape of collection container 100 or set the positions of the laser spots such that side wall 103 is positioned farther from the laser spots than a distance by which two virtual microbubbles can be arranged. As a result, strong heat convection inside the above range is less likely to be inhibited, so that microscopic objects can be collected with high efficiency.

### <Heat Dissipation Characteristics>

In general, Marangoni number Ma, which is a dimensionless number, is used as an index indicating the strength of Marangoni convection. Marangoni number Ma related to the Marangoni convection caused by the temperature difference is described as the following Mathematical formulas (1) and (2). Here, σ represents an interfacial tension of a free liquid surface of a fluid layer (sample), θ represents a temperature of the fluid layer, Δθ represents a temperature difference between an upper surface and a lower surface of the fluid layer, d represents a depth of the fluid layer, p is a viscosity coefficient of the fluid, and x is a thermal diffusion coefficient of the fluid.

$$\begin{matrix}[ Mathematical formula 1 ] & \\ Ma = \frac{Δ σ d}{μ x} & ( 1 )\end{matrix}$$

$$Δ σ = \frac{d σ}{d θ} Δ θ \qquad ( 2 )$$

From Mathematical formulas (1) and (2), it is understood that Marangoni number Ma is proportional to temperature difference Δθ between the upper part and the lower part of the sample. Therefore, in order to increase Marangoni number Ma (that is, to enhance Marangoni convection), temperature difference Δθ may be increased. The lower surface of the sample is heated by the photothermal effect of thin film 102. Therefore, in order to increase temperature difference Δθ, the temperature of the upper surface of the sample may be decreased as much as possible. Therefore, it is conceivable to enhance heat dissipation from side wall 103 of collection container 100 to the atmosphere.

In the present embodiment, cases where a metal having excellent heat dissipation characteristics is adopted and where a resin having poor heat dissipation characteristics is adopted, as the material of side wall 103 of collection container 100, are compared and examined. Specifically, an SUS was used as the metal, and acrylic was used as the resin. While the thermal conductivity of acrylic is in the range of 0.3 [W/m·K], the thermal conductivity of SUS is 16.3 [W/m·K]. The thermal conductivity of water is 0.582 [W/m·K] (10° C.).

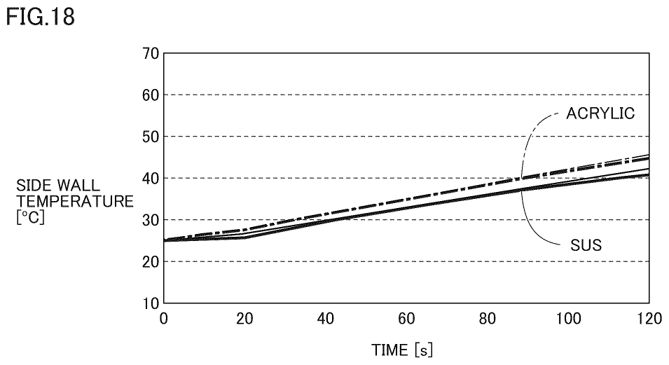

FIG. 18 is a graph showing a simulation result and an actual measurement result regarding the temperature of side wall 103 for each material of side wall 103 of collection container 100. The horizontal axis represents elapsed time. The vertical axis represents the temperature of side wall 103 of collection container 100. FIG. 18 illustrates a simulation result (see a thick solid line) and an actual measurement result (see a thin solid line) when the material of side wall 103 of collection container 100 is SUS, as well as a simulation result (see a thick one-dot chain line) and an actual measurement result (see a thin one-dot chain line) when the material of side wall 103 is acrylic.

The simulation shows that when the material of side wall 103 of collection container 100 was SUS, the temperature rise of side wall 103 was suppressed as compared with the case where the material is acrylic. The simulation results were in good agreement with the actual measurement results. The suppression of the temperature rise of side wall 103 means that heat dissipation from the sample to the atmosphere through side wall 103 is enhanced. Therefore, by adopting SUS as the material of side wall 103, temperature difference Δθ between the upper and lower sides of the sample can be increased, thereby enhancing Marangoni convection.

In addition, when the size of the laser spot (irradiation area of the laser beam) in thin film 102 is excessively reduced, the energy of the laser beam transmitted through thin film 102 increases without causing a photothermal effect, and thus there is a possibility that the lower surface of the sample cannot be heated with high efficiency. Therefore, it is desirable to make the size of the laser spot larger than the contact area between the microbubble and thin film 102. The lower surface of the sample can be heated with high efficiency by widely heating the lower surface of the sample. As a result, the Marangoni convection can be enhanced by increasing temperature difference Δθ between the upper and lower sides of the sample.

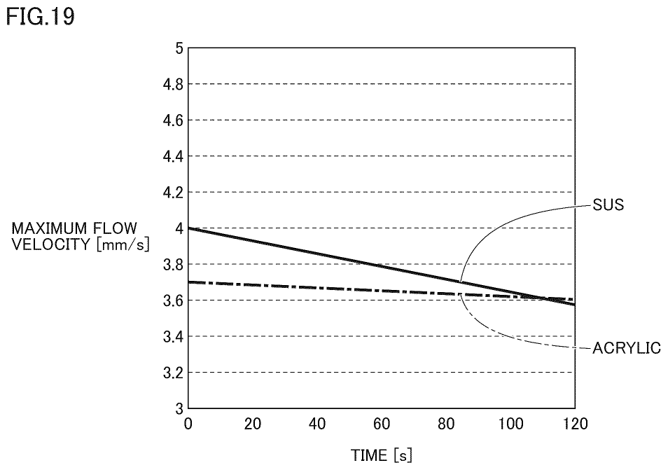

FIG. 19 is a graph showing a simulation result regarding the flow velocity of heat convection for each material of side wall 103 of collection container 100. The horizontal axis represents elapsed time. The vertical axis represents the maximum flow velocity of the heat convection within a predetermined time width. It was also confirmed by fluid simulation that Marangoni convection was enhanced by adopting SUS as the material of side wall 103.

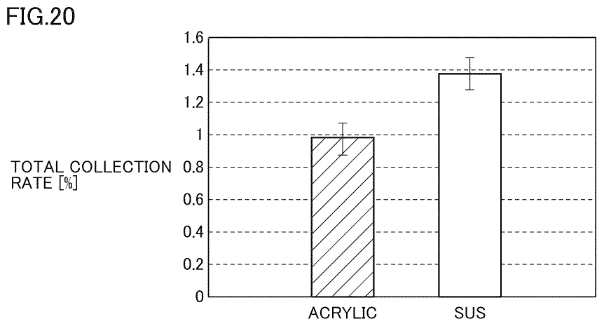

FIG. 20 is a graph showing an actual measurement result of the collected number of resin beads. The vertical axis represents a collection rate of the resin beads. The collection rate is a ratio of the number of resin beads collected in the vicinity of microbubbles to the total number of resin beads contained in the sample. As illustrated in FIG. 20, it was confirmed that when the material of side wall 103 of collection container 100 is SUS, the collection rate of resin beads was improved about 1.4 times as compared with the case where the material of side wall 103 was acrylic.

As described above, in the first embodiment, the pitch between the laser spots is set to be shorter than the distance that allows three microbubbles to be virtually arranged in a gap between two microbubbles that are actually generated. By setting the pitch to be narrow in this manner, a synergistic effect occurs in the heat convection generated around the two microbubbles, and the heat convection is enhanced. As a result, the flow velocity of heat convection can be increased as compared with the case of the single irradiation. Therefore, according to the first embodiment, a plurality of microscopic objects dispersed in a liquid can be collected with high efficiency.

### Second Embodiment

### <Influence of Shape of Gas-Liquid Interface>

In a second embodiment, a result of studying the influence of the shape and height of the gas-liquid interface on the heat convection will be described. The overall configuration of the microscopic object collecting system and the configuration of collection container 100 are similar to the configurations described with reference to FIGS. 1 to 3. Also in the second embodiment, the pitch between the laser spots is set in the same manner as in the first embodiment.

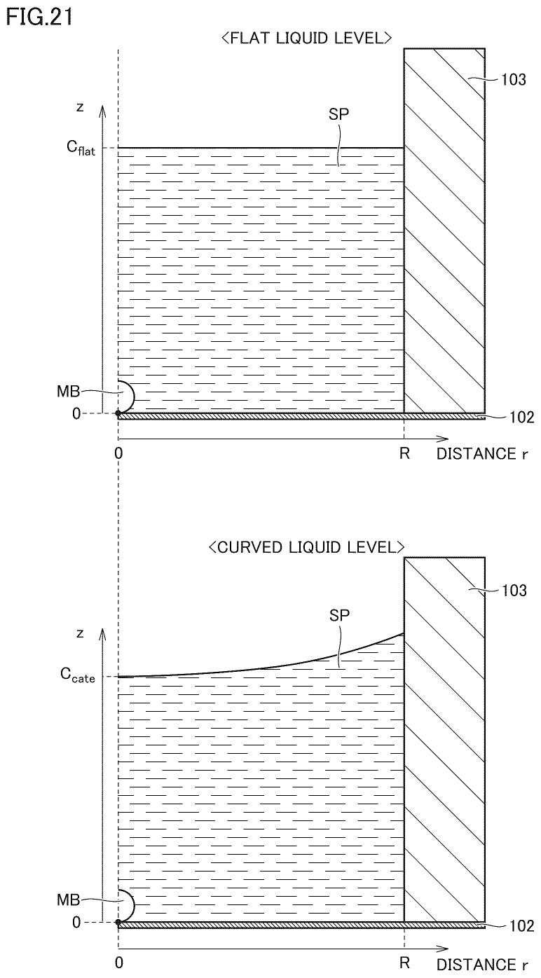

FIG. 21 is a diagram showing a simulation model for examining the influence of the shape and height of the gas-liquid interface. As shown in FIG. 21, the case where the gas-liquid interface was flat and the case where the gas-liquid interface was curved were examined. When the sample is an aqueous liquid, a flat liquid level (liquid surface means a gas-liquid interface) can be provided by using a hydrophobic material (for example, hydrophobic silicone, hydrophobic acrylic) for side wall 103, and a curved liquid level can be formed by using a hydrophilic material (for example, hydrophilic silicone, hydrophilic acrylic) for side wall 103. On the other hand, when the sample is an organic solvent, a flat liquid level can be formed by using a hydrophilic material for side wall 103, and a curved liquid level can be formed by using a hydrophobic material for side wall 103.

In this example, it is assumed that the curved liquid level is described by the catenary curve shown in the following Mathematical formula (3). Here, r represents a distance from the center of the cylindrical collection container 100, h represents the height of the liquid level with respect to the bottom surface of collection container 100, and a, b, and c are parameters defining the catenary curve.

$$\begin{matrix}[ Mathematical formula 2 ] & \\ h ( r ) = a \{ cosh ( \frac{r}{b} ) - 1 \} + c & ( 3 )\end{matrix}$$

Using Mathematical formula (3), a volume V of the sample stored in collection container 100 is calculated as in the following Mathematical formula (4).

$$\begin{matrix}[ Mathematical formula 3 ] & \\ \begin{matrix}V = {\int }_{0}^{R} d r {\int }_{0}^{2 π} d φ [ rh ( r ) ] \\ = ( c - a ) π {R}^{2} + 2 π a b [ R sinh ( \frac{R}{b} ) - b \{ cosh ( \frac{R}{b} ) - 1 \} ]\end{matrix} & ( 4 )\end{matrix}$$

When Mathematical formula (4) is solved for c, the following Mathematical formula (5) is derived. Substituting a=0 in Mathematical formula (5) represents a flat liquid level, whereas substituting a≠0 represents a curved liquid level.

$$\begin{matrix}[ Mathematical formula 4 ] & \\ c = \frac{V}{π {R}^{2}} + a - \frac{2 a b}{{R}^{2}} [ R sinh ( \frac{R}{b} ) - b \{ cosh ( \frac{R}{b} ) - 1 \} ] & ( 5 )\end{matrix}$$

Collection container 100 had an inner radius R of 7 mm and an outer radius of 9 mm. In the case of the flat liquid level, a=0 and b=3 were set. In the case of the curved liquid level, a=0.25 and b=3 were set. The radius of the microbubbles was set to 400 μm. The film thickness of thin film 102 was 0.15 mm. The absorptivity of thin film 102 with respect to a laser beam of 20 mW was set to 0.03. As hydrodynamic characteristics of the surface of collection container 100, parameters corresponding to glass were given.

Sample volume V was set in three ways of 0.3 mL, 0.5 mL, and 1 mL. A height c_{flat} of the flat liquid level at the center of collection container 100 was 1.95 mm (in the case of V=0.3 mL), 3.25 mm (in the case of V=0.5 mL), and 6.50 mm (in the case of V=1.0 mL). A height c_{cate} of the curved liquid level at the center of collection container 100 was 1.49 mm (in the case of V=0.3 mL), 2.79 mm (in the case of V=0.5 mL), and 6.04 mm (in the case of V=1.0 mL).

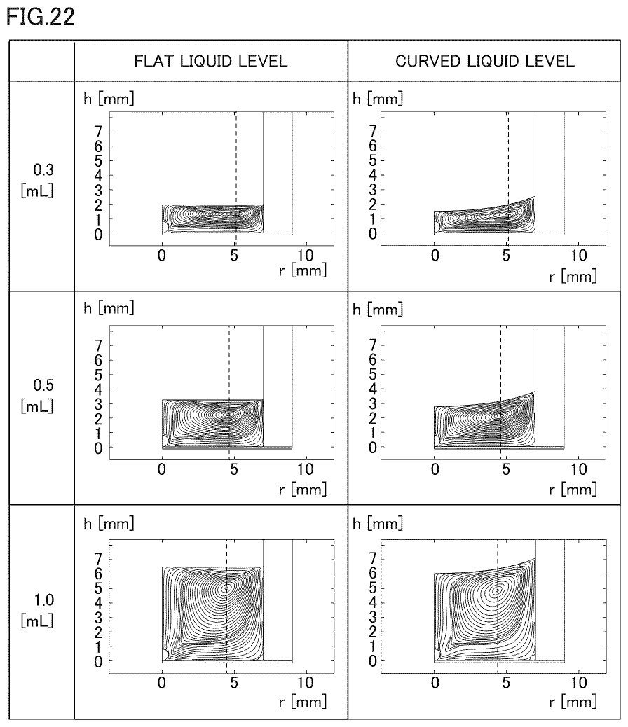

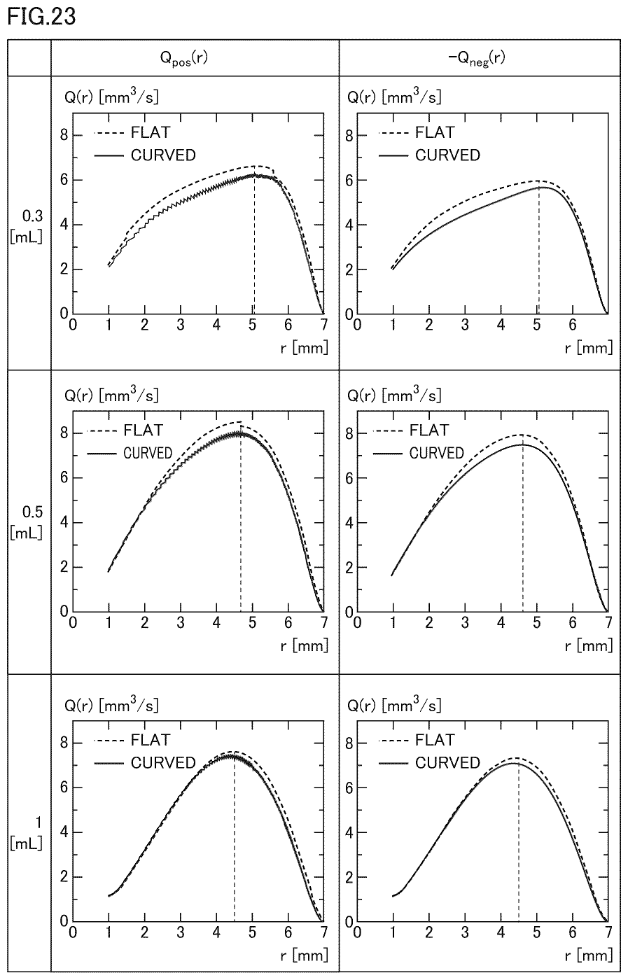

FIG. 22 is a view illustrating a fluid simulation result regarding the flow velocity of the heat convection. FIG. 22 illustrates a flow velocity field (vector field of flow velocity) of heat convection. In addition, in order to describe the relationship with FIG. 23 described later, a distance r at which the absolute value of the flow rate in the horizontal direction is the maximum and the flow rate in the height direction (vertical direction) of the sample is the minimum is indicated by a broken line.

As shown in FIG. 22, it was found that the heat convection flows along the gas-liquid interface connecting the center (r=0) of the sample and the inner wall surface (r=7 mm) of collection container 100 on both the flat liquid level and the curved liquid level. The surface area of the flat liquid level is smaller than the surface area of the curved liquid level when comparison is made between conditions where sample volumes are equal. Therefore, when convection is generated with the laser beam having the same power, the moving distance of the transported liquid is also small on the flat liquid level as compared with the curved liquid level, and the energy loss due to friction is small. Therefore, it is considered that the flow rate per unit time of the heat convection increases. In addition, the volume in the vicinity of the center of the liquid surface is larger in the flat liquid level than in the curved liquid level, and this may also affect the increase in the flow rate.

Next, a simulation result regarding a flow rate per unit time of heat convection will be described. Hereinafter, the flow rate per unit time [unit: mm³/s] of the heat convection is simply referred to as the flow rate of the heat convection.

In the present embodiment, flow rates Qₚₒₛ and Q_{neg} of the heat convection can be defined as the following Mathematical formulas (6) and (7). Here, u(r, z) is a flow velocity of the heat convection at a coordinate (r, z).

[Mathematical formula 5]

Qₚₒₛ(r)=2πr∫pos[u(r,z)]dz (6)

Q_{neq}(r)=2πr∫neg[u(r,z)]dz (7)

A function pos used to define flow rate Qₚₒₛ returns an argument x as it is when argument x is positive (more specifically, greater than or equal to 0), and returns 0 when argument x is negative (see the following Mathematical formula (8)). A function neg used to define flow rate Q_{neg} returns argument x as it is when argument x is negative (more specifically, less than or equal to 0), and returns 0 when the argument x is positive (see the following Mathematical formula (9)).

$$\begin{matrix}[ Mathematical formula 6 ] & \\ pos [ x ] = \{ \begin{matrix}x & ( 0 \leq x ) \\ 0 & ( x < 0 )\end{matrix} & ( 8 )\end{matrix}$$

$$neg [ x ] = \{ \begin{matrix}0 & ( 0 < x ) \\ x & ( x \leq 0 )\end{matrix} \qquad ( 9 )$$

Mathematical formulas (6) and (7) indicate that the flow rate of the heat convection at each distance r is calculated by integrating a flow velocity u(r) of the heat convection in the z direction (height direction). An outward flow rate (direction in which r increases) is represented by a positive value, and an inward flow rate (direction in which r decreases) is represented by a negative value.

FIG. 23 is a view illustrating a fluid simulation result regarding the flow velocity of the heat convection. The horizontal axis represents distance r [unit: mm]. The vertical axis represents flow rate Qₚₒₛ or −Q_{neg} [unit: mm³/s], i.e. an absolute value of the outward or inward flow rate along the r axis. The negative sign of −Q_{neg} is given to evaluate the absolute value and to facilitate comparison with flow rate Qₚₒₛ.

When comparison was made between conditions where the sample volumes were equal, it was found from FIG. 23 that the flow rate of the heat convection was larger at the flat liquid level than at the curved liquid level. In addition, it has been found that distance r at which flow rates Qₚₒₛ and −Q_{neg} of the heat convection are maximized and distance r at which the flow velocity in the height direction is minimized (see FIG. 22) were in good agreement with each other.

Here, since the sample was treated as an incompressible fluid, it was expected that Qₚₒₛ=−Q_{neg}, but in the actual simulation result, a slight difference occurred between Qₚₒₛ and −Q_{neg}. This difference is considered to be a numerical error. In addition, it is considered that the reason why flow rate Qₚₒₛ vibrated is that the output of the value in the vicinity of the gas-liquid interface was not stable.

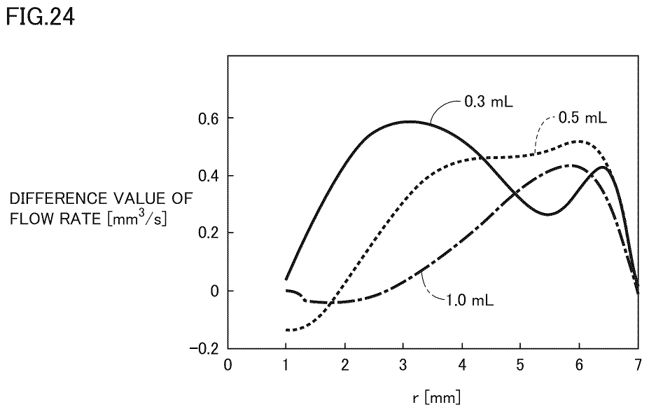

FIG. 24 is a graph for comparing flow rates of the heat convection between a flat liquid level and a curved liquid level. The horizontal axis represents distance r [unit: mm] from the center of collection container 100. The vertical axis represents a difference value [unit: mm³/s] between the value at the flat liquid level and the value at the curved liquid level, regarding an absolute value |Q_{neg}| of the flow rate of the heat convection.

When sample volume V was the largest (when V=1 mL), the difference value at the distance close to the microbubble (a distance close to the center of the sample, for example, a distance within a range of r=1 mm to 3 mm) was almost 0. This is considered to be because the influence of the shape of the gas-liquid interface on heat convection (particularly Marangoni convection) is relatively small because the distance between the gas-liquid interface and the microbubbles is long when sample volume V is the largest.

On the other hand, when sample volume V was smaller (when V=0.3 mL, 0.5 mL), the difference value at the distance close to the microbubble was larger than that when sample volume V was the largest (V=1 mL). That is, absolute value |Q_{neg}| of the flow rate at the flat liquid level was significantly larger than absolute value |Q_{neg}| of the flow rate at the curved liquid level. The reason for this is described as follows. When considering a cylindrical minute region in a range from distance r to r+dr, the volume of the minute region having a flat liquid level is larger than the volume of the minute region having a curved liquid level at a distance close to the microbubble. As the volume of the minute region increases, the flow rate flowing through the minute region also increases. Therefore, it is considered that by making the shape of the gas-liquid interface flat, the flow rate of the heat convection can be increased as compared with the case where the gas-liquid interface is curved.

As described above, also in the second embodiment, as in the first embodiment, the pitch between the laser spots is set to be shorter than the distance that allows three microbubbles to be virtually arranged in a gap between two microbubbles that are actually generated. As a result, a plurality of microscopic objects dispersed in the liquid can be collected with high efficiency. Furthermore, in the second embodiment, the sample volume is adjusted and the wettability of side wall 103 of collection container 100 is set so that the gas-liquid interface is flat. When the gas-liquid interface is flat, a flow line of the heat convection is shorter than that when the gas-liquid interface is curved, and therefore the flow rate of the heat convection flowing along the gas-liquid interface can be increased. When the gas-liquid interface is flat, a volume of the minute region within the same distance from the center of the sample (within the distance from r to r+dr) is large as compared with the case where the gas-liquid interface is curved, and therefore the flow rate of heat convection at distance r can be increased. Hence, according to the second embodiment, a plurality of microscopic objects dispersed in a liquid can be collected with higher efficiency.

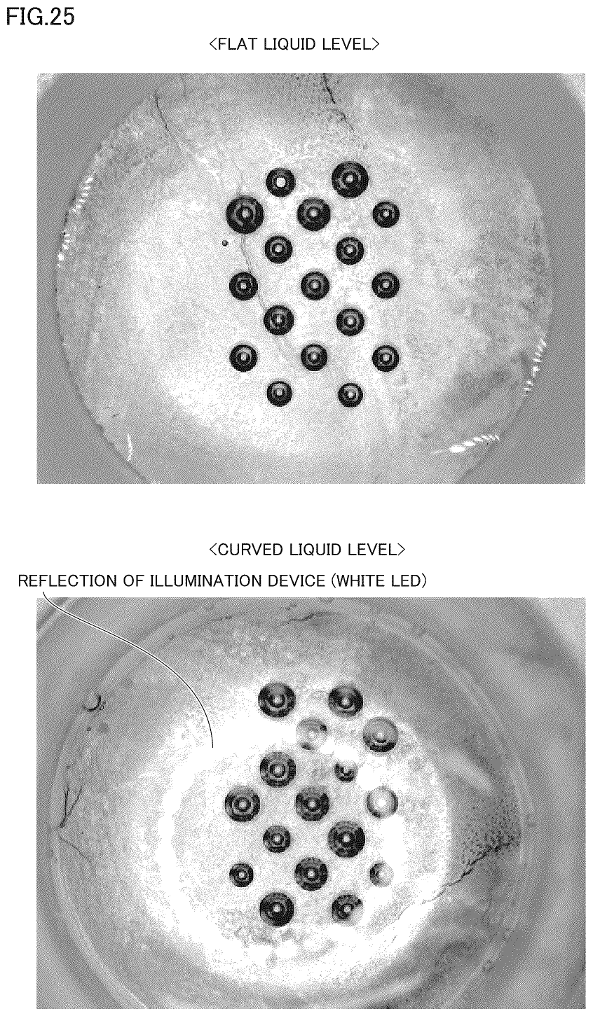

In subsequent FIGS. 25 to 27, a result when the resin beads are actually collected will be described. FIG. 25 is an image taken by photographing the flat liquid level and the curved liquid level. All of these images were obtained for sample volume V=1 mL. The flat liquid level was provided by side wall 103 of hydrophobic silicone and the curved liquid level was provided by side wall 103 of hydrophilic acrylic. In either case, the inner diameter of collection container 100 was 14 mm. A white LED arranged in a circular shape was used as illumination device 9. It can be seen that light from the white LED was reflected on the liquid level at the curved liquid level by the curved liquid level functioning as a concave lens, whereas such light was not taken in the image at the flat liquid level.

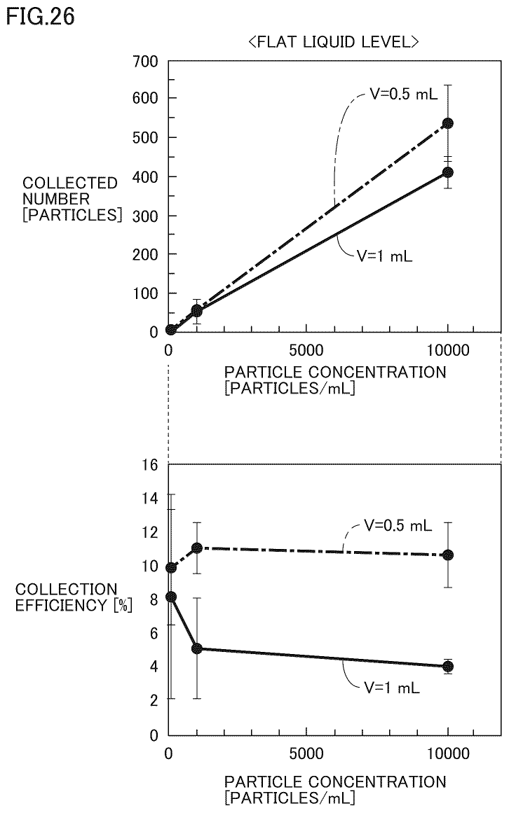

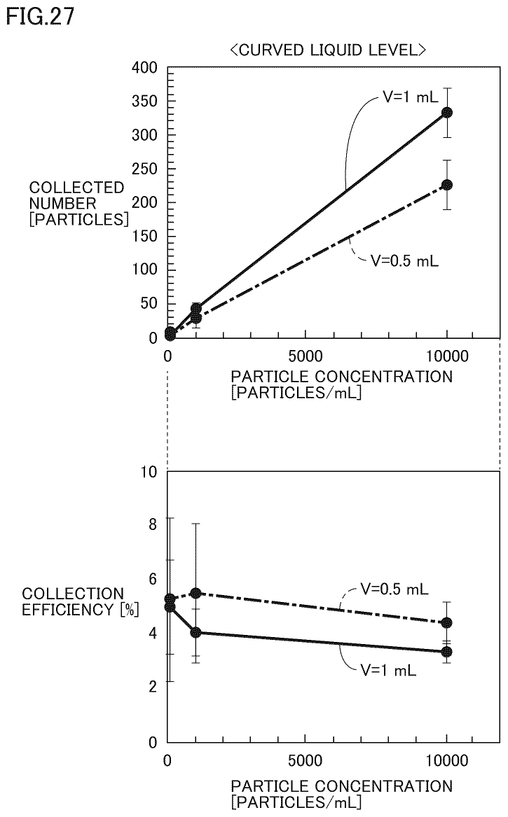

FIG. 26 is a graph showing an example of measurement results of the collected number of resin beads and collection efficiency on the flat liquid level. Sample volume V was set in two ways of 0.5 mL and 1 mL. The horizontal axis represents the particle concentration (concentration of resin beads introduced into the sample) [unit: particles/mL]. The upper vertical axis represents the total collected number of the resin beads collected in all the laser spots. The lower vertical axis represents the collection efficiency. The same applies to FIG. 27.

In the case of the flat liquid level, when sample volume V was small (V=0.5 mL), the collected number of resin beads increased and the collection efficiency also increased as compared with the case where sample volume V was large (V=1 mL). This tendency was particularly remarkable when the particle concentration was high (10,000 particles/mL).

FIG. 27 is a graph showing an example of measurement results of the collected number of resin beads and collection efficiency on the curved liquid level. In the case of the curved liquid level, unlike the flat liquid level, the collected number of resin beads was reduced when sample volume V was small, as compared with the case where sample volume V was large. On the other hand, also in the case of the curved liquid level, similarly to the flat liquid level, the collection efficiency of the resin beads increased when sample volume V was small, as compared with the case where sample volume V was large.

It should be understood that the embodiments disclosed herein are illustrative in all respects and not restrictive. The scope of the present disclosure is defined not by the description of the above embodiments but by the claims, and is intended to include meanings equivalent to the claims and all modifications within the scope.

### REFERENCE SIGNS LIST

1: sample stage, 2: sample supply device, 3: light source stage, 4: laser module, 41: base material, 42: surface emitting element, 421: light emitting region, 43: bonding member, 44: optical waveguide, 441: core, 442: cladding, 45: lens, 46: laser device, 47: laser module, 5: cooling device, 6: adjustment mechanism, 7: power supply, 8: imaging device, 9: illumination device, 10: controller, 100: collection container, 101: substrate, 102: thin film, 103: side wall, 901, 902: collecting system

1: A microscopic object collecting method for collecting a plurality of microscopic objects dispersed in a liquid, the method comprising:
irradiating a photothermal conversion region with a plurality of beams, the photothermal conversion region being provided on a bottom surface of a container containing the liquid, the plurality of beams being separated from each other; and
heating the liquid with the plurality of beams to generate a plurality of microbubbles corresponding to the plurality of beams and to generate heat convection in the liquid,
wherein an interval between adjacent two beams of the plurality of beams is narrower than a distance that allows three larger microbubbles to be virtually arranged in a gap between two microbubbles corresponding to the two beams, each of the three larger microbubbles being the larger one of the two microbubbles.

2: The method according to claim 1, wherein the interval is narrower than a distance that allows one of the three larger microbubbles to be virtually arranged in the gap.

3: The method according to claim 1, wherein a distance between an irradiation position on the photothermal conversion region with each of the plurality of beams and a side wall of the container is longer than a diameter of a corresponding one of the plurality of microbubbles.

4: The method according to claim 1, wherein a thermal conductivity of the side wall of the container is larger than a thermal conductivity of the liquid.

5: The method according to claim 1, wherein an irradiation area on the photothermal conversion region with each of the two beams is larger than a contact area between a corresponding one of the two microbubbles and the photothermal conversion region.

6: The method according to claim 1, further comprising:
storing the liquid in the container, prior to the irradiating, such that a gas-liquid interface between the liquid and a surrounding gas is flat.

7: A microscopic object collecting system that collects a plurality of microscopic objects dispersed in a liquid, the system comprising:
a holder that holds a storage container for the liquid, the storage container having a bottom surface provided with a photothermal conversion region;
a light source that emits a plurality of beams; and
an optical system configured to irradiate the photothermal conversion region with the plurality of beams that are separated from each other,
wherein by heating the liquid with the plurality of beams, a plurality of microbubbles are generated at irradiation positions of the plurality of beams and heat convection is generated in the liquid, and
the optical system is configured such that an interval between adjacent two beams of the plurality of beams is narrower than a distance that allows three larger microbubbles to be virtually arranged in a gap between two microbubbles generated by the two beams, each of the three larger microbubbles being the larger one of the two microbubbles.
